(19) United States
(12) Patent Application Publication
(10) Pub. No.: US 2012/0115891 A1
(43) Pub. Date: May 10, 2012

(54) METHOD FOR THE INDUCTION OF A REWARD RESPONSE BY MODULATION OF DOPAMINERGIC SYSTEMS IN THE CENTRAL NERVOUS SYSTEM

(75) Inventor: Dennis Jones, Shelburne, VT (US)

(73) Assignee: SOMALABS INC., South Burlington, VT (US)

(21) Appl. No.: 13/264,586

(22) PCT Filed: Apr. 16, 2010
(86) PCT No.: PCT/US10/31430

§ 371 Date: Jan. 3, 2012

Related U.S. Application Data
(60) Provisional application No. 61/170,234, filed on Apr. 17, 2009.

(51) Int. Cl. A61K 31/198; A61P 3/04; A61K 31/4747; A61P 25/00; A61K 31/473; A61K 31/522

(52) U.S. Cl. 514/263.34; 514/567; 514/284; 514/278

(57) ABSTRACT

A method of modulating the dopaminergic system of the central nervous system comprising administering to a subject a dopamine precursor and/or a dopamine agonist in an amount effective to induce a reward response in the subject is described, as are related compositions.

### CROSS-REFERENCE TO RELATED APPLICATIONS

This application claims priority to U.S. Provisional Application No. 61/170,234, filed Apr. 17, 2009, the entire contents of which are herewith incorporated by reference.

### BACKGROUND OF THE INVENTION

This invention relates to the field of pharmacology and behavior modification.

It has long been speculated that the performance of a pleasurable act or activity could modify neurochemical function of various parts of the central nervous system, often reinforcing the pleasurable effects of the act or activity and causing it to be repeated more frequently. Such reinforcement may be considered as a subconscious method of conditioning, which is self propagating to the extent that it can, in some cases, result in apparently permanent changes in behavior. In simple terms, a human performs an act “A” which changes levels or proportions of neurotransmitters in certain parts of the central nervous system. Such changes may be localized or more widespread, and by increasing or reducing the extent of receptor occupancy they may affect perception, behavior or mood. This positive, self-reinforcing response to a pleasurable act, though it may be difficult to quantify, can be described as a reward response or a reward reaction, and it has been considered to play a role in various types of physiological and pathological conditions. For example, it has been reported that the reward response is diminished in attention deficit hyperactivity disorder (ADHD), and most recently, that it is blunted in obesity. The role of the reward response in various types of addiction or habituation has also been discussed, though it appears that it may be purely peripheral, and even if involved, not of prime importance, in the use of mind-altering drugs ranging from alcohol and tobacco to cocaine.

With the advent of increasingly more sophisticated methods of examining the function of the central nervous system, such as positron emission tomography and functional magnetic resonance imaging (fMRI), it has been shown that the reward response is triggered when dopaminergic systems in the brain, particularly the mesolimbic system, are activated as shown in FIG. 1. Activation of such systems means that the levels of occupancy of the dopamine receptors in the tissues are increased.

The role of the reward response in eating behavior remains unclear. On the one hand, it has been hypothesized that an exaggerated or pathological reward response might occur in response to eating, and that this might result in a self-sustaining loop. On the other hand, it has most recently been shown that the obese have a blunted response to food, and therefore are required to consume greater quantities of food in order to achieve even a normal reward response.

It is likely that differences in methodology may cause some of the contradictory findings in the role of the reward response in eating behavior, but these contradictory findings do all result in the conclusion that the reward response is pathological in disorders of eating behavior.

### SUMMARY OF THE INVENTION

In one embodiment, the invention is directed to a method of modulating the dopaminergic system of the central nervous system, the method comprising administering to a subject a dopamine precursor and/or a dopamine agonist in an amount effective to induce a reward response in the subject. A reward response simulates a desired state of being in a subject.

In one embodiment, this desired state of being is a reduced interest in food, a reduced craving for food or a feeling of having already eaten. In such method, the administering is oral and the desired state of being is achieved within 90 minutes, so that administration should take place long enough before a planned meal to allow development of this desired state of being.

In one embodiment, the method of the invention involves administering both a dopamine precursor and a dopamine agonist. The dopamine precursor may be L-DOPA and the dopamine agonist may be an aporphine alkaloid. In one embodiment, the dopamine precursor is L-DOPA and the dopamine agonist is aporphine alkaloid. In another embodiment the L-DOPA and aporphine alkaloid are extracted from plants. In one embodiment, the L-DOPA is extracted from Mucuna pruriens. In another embodiment, the aporphine alkaloid is extracted from Nelumbo nucifera.

In another embodiment, the invention is a composition for reducing a subject's desire to eat comprising an effective amount of a dopamine precursor, a dopamine agonist and pharmaceutically acceptable excipients. Such composition may comprise L-tyrosine (USP), an extract from Mucuna pruriens, an extract from Nelumbo nucifera, an extract from Citrus aurantium and an extract from Griffonia simplicifolia. In a preferred embodiment, the invention comprises 150 mg of L-tyrosine (USP), 100 mg of extract from Mucuna pruriens, 100 mg of leaf extract from Nelumbo nucifera, and 40 mg of extract from Citrus aurantium, wherein 25% by weight of said Mucuna pruriens extract is L-DOPA, 8% by weight of said Nelumbo nucifera leaf extract is aporphine alkaloids; 30% by weight of said Citrus aurantium is one or more of an alkaloid selected from the group consisting of synephrine, octopamine, hordenine, tyramine and N-methyl-tyramine, and 25% by weight of said Griffonia simplicifolia extract is 5-hydroxytryptophan.

In another embodiment, the composition comprises an extract from Mucuna pruriens, an extract from Camellia sinensis, and a leaf extract from Nelumbo nucifera. In a preferred embodiment, the composition comprises 300 mg of Mucuna pruriens, 250 mg of Camellia sinensis and 200 mg of Nelumbo nucifera, wherein 25% by weight of said Mucuna pruriens is L-DOPA, 36% by weight of said extract from Camellia sinensis is caffeine and 45% by weight is catechols, and 8% by weight of said Nelumbo nucifera leaf extract is aporphine alkaloids.

The composition of the invention can be in any pharmaceutically acceptable form but preferably in capsules or tablets.

### BRIEF DESCRIPTION OF THE DRAWINGS

FIG. 1 is a schematic of the human brain.

FIG. 2 is a schematic that shows the conversion process of precursors of dopamine to L-DOPA, dopamine and adrenaline.

FIG. 3 is a schematic that shows the rapid conversion of L-DOPA into dopamine.

FIG. 4 shows the chemical formulae for nuciferine and related alkaloid extracted from Nelumbo nucifera (Sacred Lotus).

FIG. 5a shows a dopamine substructure within an aporphine alkaloid required for dopamine agonist activity.

FIG. 5b shows the dopamine substructure required for dopamine agonist activity of dopamine itself.

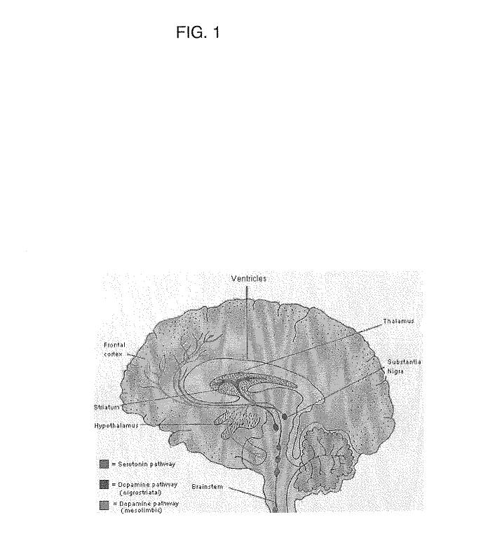

### DETAILED DESCRIPTION OF THE INVENTION

It has now surprisingly been found that the reward response can be manipulated by the administration of dopamine precursors and/or dopamine agonists. Thus, in one embodiment, the invention is directed to a method of modulating the dopaminergic system of the central nervous system, the method comprising administering to a subject a dopamine precursor and/or a dopamine agonist in an amount effective to induce a reward response in the subject. Although the inventor does not want to be bound by any single theory of how the dopaminergic system is modulated according to this invention, it is believed that modulation occurs when dopamine receptors in the brain are bound or occupied by dopamine or dopamine agonists. A “reward response” simulates a desired state of being in a subject. For instance, the desired state of being may be the satisfaction felt upon intake of caffeine, nicotine, alcohol, cocaine or some other pleasure-giving drug or substance. Thus, the purpose of the invention is to create or simulate in a subject the state of being associated with ingesting a given substance without the subject actually having ingested the substance. This is a “reward response,” which also could be called a “premature” or a “false positive” reward response.

In one embodiment, the method of the invention is used to facilitate weight loss, in that the invention causes beneficial changes in eating behavior. Within the context of food consumption, the term “reward response,” means that the subject has a reduced interest in food, a reduced craving of food or a feeling of already having eaten. Consequently, the desire to eat more food is reduced or eliminated. Consequently, weight loss occurs with reduced intake of food.

It is possible to modulate the dopaminergic system by triggering a reward response or premature or false positive reward response by either increasing availability of dopamine in the dopaminergic systems in the brain, an effect that can be achieved by increasing the levels of precursors of dopamine in the brain, or by the administration of a so-called dopamine agonist, that is, a substance which mimics the action of dopamine and can occupy dopamine receptors. The combination of a dopamine precursor with a dopamine agonist has surprisingly proved particularly effective in triggering a reward response which is capable of modifying eating behavior, provided that such a response can be achieved before food is actually eaten.

The main, and conventional, precursors of dopamine are the essential amino acids L-phenylalanine and L-tyrosine. These are present in most proteins, but may also be administered as the free amino acids. However, they require conversion in the body, and the conversion process under normal physiological conditions is relatively inefficient and slow. It is therefore desirable to administer the dopamine precursor as L-3,4-dihydroxyphenylalanine, also known as L-DOPA, which is rapidly and efficiently converted into dopamine in the central nervous system and other tissues.

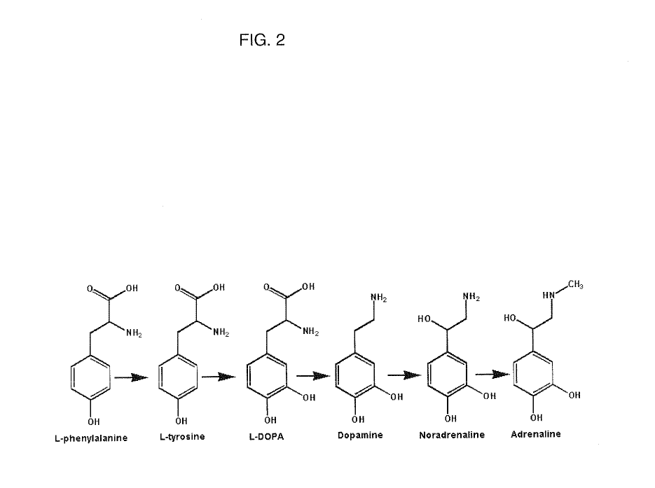

FIG. 2 shows the conversion process schematically.

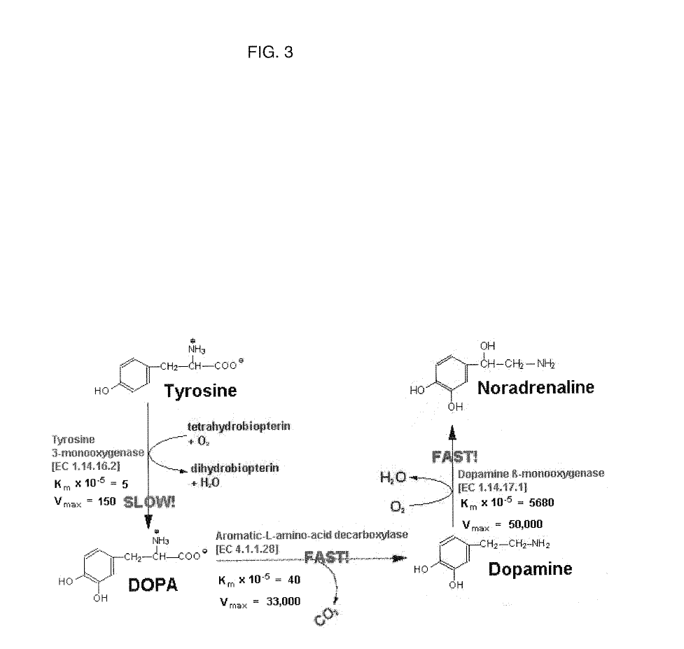

More importantly, the conversion of L-DOPA into dopamine is rapid compared with the preceding conversion steps, as illustrated in FIG. 3.

While the present invention relates to a method for providing increased dopamine availability in the central nervous system, and thus increased occupation of dopamine receptors, it should be noted that noradrenaline is the main mediator of thermogenesis in peripheral tissues. Thus, the provision of a dopamine precursor as L-DOPA also serves to increase the resting metabolic rate in peripheral tissues, and therefore assists with body weight control via this mechanism.

Dopamine agonists are well-known in medicine, and include bromocriptine, cabergoline, pergolide, pramiprexole, ropinirole, apomorphine and rotigotine. These drugs are relatively non-selective for the various subsets of dopamine receptors, and are used for the treatment of Parkinson's disease, certain pituitary tumors, restless leg syndrome and some types of sexual dysfunction. Drugs that are selective for a particular type of dopamine receptor, such as fenoldopam (selective for D1 receptors) are also known.

There are at least 5 types of dopamine receptor in the human body, classified as D1, D2, D3, D4 and D5. Putatively, receptors of D6 and D7 type may also exist. According to this invention, relatively non-selective dopamine agonists acting on receptors of D1 and D2 type are preferred, since both D1 and D2 type receptors are involved in the reward response. Thus, the preferred dopamine agonists of the invention bind and are preferably selective for the D1 and D2 receptors.

U.S. Pat. No. 4,939,174 describes dopamine-fatty acid conjugates that are lipophilic complexes of dopamine itself and which readily pass through the blood brain barrier. The development of a compound that would pass through the blood brain barrier was the main objective of the invention for U.S. Pat. No. 4,939,174. Uses of this complex include appetite suppression, although this patent does not describe modulation of the reward response to modify eating behavior. It is well-known that dopamine is converted into norepinephrine in the tissues and that norepinephrine acts on a center in the brain to suppress hunger. Consequently, it would have been expected that the disclosed complex could be used for appetite suppression. However, this is totally unrelated to the modulation of the reward response of the present invention.

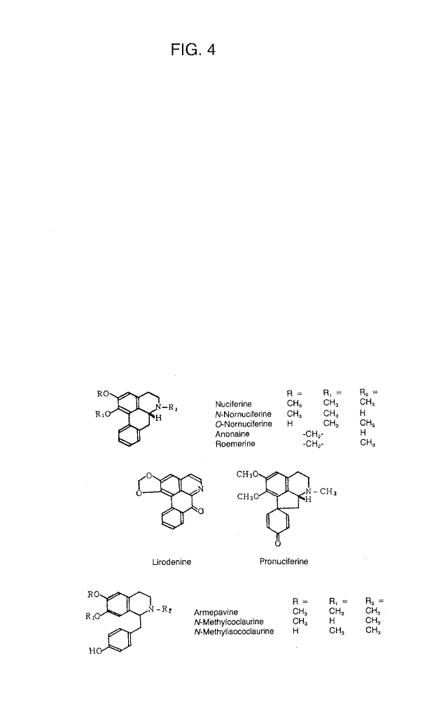

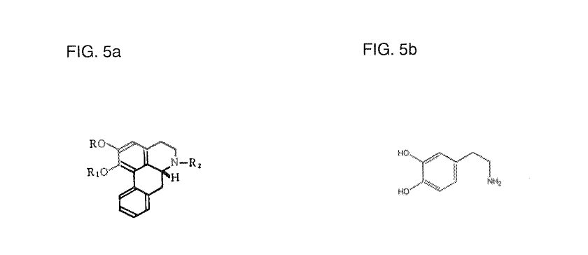

Naturally occurring dopamine agonists are also known, and are preferred. While substances that are dopamine agonists are present in some Convolvulaceae (Ipomoea, Rivea and Argyreia species), for example, lysergic acid amide, such dopamine agonists, or other active substances in these plant species, may exert effects of hallucinogenic nature, and are a less viable option from a safety perspective. Dopamine agonists have also been demonstrated in Cactaceae, including Lophophora species, but are again burdened with other central nervous system actions that make them less desirable for use. It has, however, been discovered that various species of Nelumbo, and in particular Nelumbo nucifera, contain aporphine type alkaloids in high concentrations without the presence of other substances whose safety might be suspect. Other plant derived dopamine agonists suitable for use in the methods and compositions of the invention are described in Huang, K., The Pharmacology of Chinese Herbs (2^{nd} Edition), CRC Press, Boca Raton, Fla. (1999), the relevant parts of which are hereby incorporated by reference. Concentrated extracts of the leaf, root or seed on Nelumbo nucifera, also known as Sacred Lotus, are thus preferred. Such extracts contain nuciferine and related alkaloids (FIG. 4) which are sufficiently lipophilic to cross into the brain, and contain the dopamine substructure (FIGS. 5a and 5b) required for agonist activity. Examples of other natural dopamine agonists include but are not limited to extracts of Zizyphus vulgaris, Zizyphus, jujuba and Cananga odorata. The dopamine agonists of the present invention are extracted according to methods well known in the art and many are commercially available. Also within the scope of the present invention are modified plant extracts, which are modified according to methods known to the skilled artisan (e.g., through fermentation or contact with enzymes). Thus, the compositions and methods of the invention could comprise both modified and/or unmodified plant extracts.

The invention therefore relates to the administration by any route but preferably by oral route of a substance, which will result in a greater occupancy of dopamine receptors in the dopaminergic areas of the human central nervous system, and particularly in the mesolimbic system, whereby a reward response is initiated, such that the consumer is incapable of mounting a further reward response when confronted with food or the promise of food, and thus lacks the motivation to eat to excess. Such a substance is, or in the case of extracts of natural products, comprises, either a precursor of dopamine or a substance that mimics the action of dopamine in the central nervous system or a combination of both.

In a preferred embodiment of this invention, use is made of a natural source of L-DOPA (L-3,4-dihydroxyphenylalanine), such as an extract of the Velvet Bean (Mucuna pruriens), whereby sufficient L-DOPA is administered to create a relative surplus of dopamine in the central nervous system. In general, and allowing for individual variations, the amount of L-DOPA administered may range from 10 mg to 2500 mg per dose, though most subjects respond well to doses of 50 mg to 500 mg.

In another preferred embodiment, the invention is directed to the administration of a natural source of aporphine alkaloids, of the general structure shown in FIG. 5a, such as an extract of the leaf, root or seed of the Sacred Lotus (Nelumbo nucifera). In general, and allowing for individual variations, the amounts of mixed aporphine alkaloids administered may range from 1 mg to 100 mg, though most subjects respond well to doses of 5 mg to 50 mg.

In yet another preferred embodiment, both a natural source of L-DOPA and a natural source of aporphine alkaloids are administered concomitantly, whereby consumers receive 50 mg to 500 mg L-DOPA and 1 mg to 100 mg aporphine alkaloids per dose. In general, humans respond well to the co-administration of these active agents in the form of Velvet Bean extracts and Sacred Lotus leaf, root or seed extracts when the amounts of L-DOPA and aporphine alkaloids are in the ranges of 50 mg to 500 mg and 2 mg to 50 mg, respectively. In a preferred embodiment, the amount of the L-DOPA is from 50 to 150 mg and the amount of the aporphine alkaloid is from 16 to 32 mg. Such a combination in a suitable pharmaceutical form, which may be a capsule, tablet, softgel capsule or powder for reconstitution to a drink is particularly effective when given at least 30 minutes before a planned meal or snack, but not more than 90 minutes before the planned meal or snack. The point of the timing of the composition(s) of the invention is to give the active ingredients a chance to circulate and reach the target receptors before the subject is exposed to food or whatever is normally giving them a reward response. Accordingly, the subject has a reward response that mimics the response that they would have received by ingesting the food or other substance they wanted. The timing might need to vary somewhat depending upon the subject.

The composition of the present invention can be formulated according to methods known to the skilled artisan. The preferred formulation is for oral delivery. The ingredients of the compositions of this invention are contained in acceptable excipients and/or carriers for oral consumption. The actual form of the carrier, and thus, the composition itself, is not critical. The carrier may be a liquid, gel, gelcap, capsule, powder, solid tablet (coated or non-coated), tea, or the like. The composition is preferably in the form of a tablet or capsule and most preferably in the form of a hard gelatin capsule. Suitable excipient and/or carriers include maltodextrin, calcium carbonate, dicalcium phosphate, tricalcium phosphate, microcrystalline cellulose, dextrose, rice flour, magnesium stearate, stearic acid, croscarmellose sodium, sodium starch glycolate, crospovidone, sucrose, vegetable gums, lactose, methylcellulose, povidone, carboxymethylcellulose, corn starch, and the like (including mixtures thereof). Preferred carriers include calcium carbonate, magnesium stearate, maltodextrin, and mixtures thereof. The various ingredients and the excipient and/or carrier are mixed and formed into the desired form using conventional techniques. The tablet or capsule of the present invention may be coated with an enteric coating that dissolves at a pH of about 6.0 to 7.0. Suitable enteric coatings that dissolve in the small intestine but not in the stomach are cellulose acetate phthalate or methacrylic acid copolymers. Further details on techniques for formulation for and administration may be found in the latest edition of Remington's Pharmaceutical Composition Sciences (Maack Publishing Co., Easton, Pa.).

It is understood by those of skill in the art that other ingredients can be added to those described herein, for example, fillers, emulsifiers, flavors, coloring agents, sweeteners, preservatives, etc. for the processing or manufacture of the composition of the present invention.

### EXAMPLES

The invention is illustrated by means of the following examples, which are non-limiting. Percentages are calculated by weight, unless otherwise indicated. Reference may be made to trade names for components. Inventors do not intend to be limited to the materials covered by various trade names and intend to include equivalents of the materials under such trade names.

### Example 1

A blend of ingredients was made and capsules (white size 0) were filled according to the following formulation:

| | Component: | Mg/capsule: | % |
|---|---|---|---|
| | L-tyrosine (USP) | 150.00 | 33.34 |
| 1) | Velvet bean (Mucuna pruriens) | 100.00 | 22.22 |
| | extract | | |
| 2) | Sacred lotus (Nelumbo nucifera) | 100.00 | 22.22 |
| | extract | | |
| 3) | Bitter orange (Citrus aurantium) | 40.00 | 8.89 |
| | extract | | |
| 4) | Griffonia (Griffonia simplicifolia) | 40.00 | 8.89 |
| | extract | | |
| | Magnesium stearate | 10.00 | 2.22 |
| | Silica | 10.00 | 2.22 |
| | | 450.00 | 100.00 |

1) Velvet bean extract contained 25% L-DOPA; 100 mg provided 25 mg L-DOPA.
2) Nelumbo nucifera leaf extract, 8% alkaloids; 100 mg provided 8 mg mixedaporphine alkaloids.
3) Bitter orange extract at 30% alkaloids (synephrine, octopamine, hordenine,tyramine, N-methyl-tyramine.
4) Griffonia extract was 25% 5-hydroxytryptophan.

The resultant capsules provided, per capsule, 175 mg of dopamine precursors and 8 mg of dopamine agonists.

### Example 2

A blend of ingredients according to the following formulation was made, and compressed to tablets weighing 1000 mg each:

| | Component: | Mg/tablet: | % |
|---|---|---|---|
| 1) | Velvet bean (Mucuna pruriens) | 300.00 | 30.000 |
| | extract | | |
| 2) | Green tea (Camellia sinensis) | 250.00 | 25.000 |
| | extract | | |
| 3) | Sacred lotus (Nelumbo nucifera) | 200.00 | 20.000 |
| | extract | | |
| | Starch USP | 130.00 | 13.000 |
| | Croscarmelose sodium | 50.00 | 5.000 |
| | Microcrystalline cellulose | 50.00 | 5.000 |
| | Magnesium stearate | 10.00 | 1.000 |
| | Silica | 10.00 | 1.000 |
| | Total weight excluding film coat | 1000.00 | 100.00 |

1) Velvet bean extract contained 25% L-DOPA; 300 mg provided 75 mg L-DOPA.
2) Green Tea extract was 36% caffeine and 45% catechols
3) Sacred lotus was the Nelumbo nucifera leaf extract, 8% alkaloids; 200 mg provided 16 mg mixed aporphine alkaloids.

After compression, the tablets were film-coated with hydroxypropylmethylcellulose.

The disintegration time of these tablets in simulated gastric juice was 30 minutes. They provided, per tablet, 75 mg of dopamine precursors as L-DOPA and 16 mg dopamine agonists.

### Example 3

Male subject DJ took 5 capsules prepared as described in Example 1, prior to undergoing fMRI of the central nervous system. There was a brief period of mild euphoria after about 45 minutes, lasting 15-30 minutes, accompanied by a subjective feeling of well-being which persisted for about 2 hours. When confronted with a meal 3 hours after taking the capsules, the subject had a distinct lack of appetite and lack of interest in eating.

### Example 4

Female subject ADG, who had been gaining weight, took 2 capsules (prepared as described in Example 1) 3 times daily for 28 days, 45 minutes before meals. Per dose intake consisted of 350 mg dopamine precursors (of which 25 mg was L-DOPA) and 16 mg dopamine agonists. During this time, the subject ceased gaining weight and reported that she had lost interest in her favorite foods and snacks. She then ceased taking the capsules. One week after ceasing use of the capsules, she reported that she was again gaining weight and had recovered her interest in her favorite foods.

### Example 5

Five female subjects took 2 capsules (prepared as described in Example 1) 3 times daily for 14 days, 45 minutes before meals. They continued their normal eating habits during this period. Body weight and blood pressures were recorded on day 0, before use of the capsules commenced, and again on days 7 and 14. No significant changes in either diastolic or systolic blood pressure were seen. Body weight changes were:

| | Body weight (kg) | | | Change in weight (kg) |
|---|---|---|---|---|
| Subject | Day 0 | Day 7 | Day 14 | (Day 0 to Day 14) |
| HB | 73.9 | 71.8 | 71.4 | −2.5 |
| SW | 63.6 | 62.7 | 61.4 | −2.2 |
| RE | 75.0 | 75.4 | 74.5 | −0.5 |
| BS | 58.9 | 57.0 | 55.4 | −3.5 |
| DH | 64.1 | 64.5 | 62.7 | −1.4 |

The treatment therefore resulted in weight loss. Per dose intake consisted of 350 mg dopamine precursors (of which 25 mg was L-DOPA) and 16 mg dopamine agonists.

### Example 6

Female subjects HB and CJ took 2 capsules (prepared as described in Example 1) 3 times daily for 14 days, 45 minutes before meals. They continued their normal eating habits during this period. On days 0, 1, 7 and 14 they completed Linear Rating Scales to quantify mood, vitality, well-being, hunger, satiety and interest in food. The Linear Rating Scales had a range from 0 to 15, with the scales correlating to the following extremes, where higher values indicate positive effects on the scales for mood, vitality, wellbeing, hunger and satiety, and lower values indicate positive effects on the scale for interest in food:

Mood: 0=poor, 15=excellent
Vitality (feeling of energy): 0=poor, 15=excellent
Well-being: 0=poor, 15=excellent
Hunger: 0=very hungry, 15=not at all hungry
Satiety: 0=do not feel full quicker, 15=feel full very quickly
Interest in food: 0=not at all interested, 15=very much interested in food.

Results obtained are tabulated below:

| Subject | Parameter | Day 0 | Day 1 | Day 7 | Day 14 |
|---|---|---|---|---|---|
| HB | Mood | 10.0 | 10.5 | 7.5 | 5.5 |
| | Vitality | 2.5 | 11.5 | 10.0 | 5.5 |
| | Well-being | 12.0 | 10.0 | 12.0 | 8.5 |
| | Hunger | 7.5 | 4.0 | 5.5 | 8.5 |
| | Satiety | 2.0 | 9.5 | 11.0 | 8.0 |
| | Interest in food | 9.5 | 4.0 | 6.0 | 7.0 |
| CJ | Mood | 5.0 | 9.5 | 7.0 | 9.0 |
| | Vitality | 5.0 | 8.0 | 7.0 | 9.0 |
| | Well-being | 5.0 | 8.0 | 7.0 | 8.5 |
| | Hunger | 10.5 | 7.5 | 7.0 | 7.5 |
| | Satiety | 10.5 | 8.0 | 7.0 | 9.0 |
| | Interest in food | 12.0 | 11.5 | 7.5 | 9.0 |

The results indicate that the treatment had an effect mostly on interest in food, which decreased in both subjects, interpreted as an effect on the reward response, namely that the treatment elicited a reward response which partially substituted for the reward response created by food.

### Example 7

Six overweight subjects (5 female, 1 male) took 2 tablets (prepared as described in Example 2) 3 times daily for 14 days, 45 minutes before meals. Per dose intake provided 150 mg of dopamine precursors (as L-DOPA) and 32 mg of dopamine agonists. They continued their normal eating habits during this period. They completed Linear Rating Scales on day 0, before use of the tablets commenced, and again on days 1, 7 and 14. Evaluation and statistical analysis of the Linear Rating Scales showed significant effects on all subjective parameters quantified. The Linear Rating Scales used were modified from those used in Example 6, and had a range from 0 to 15, with the scales correlating to the following extremes, where higher values indicate positive effects on the scales for mood, vitality, well-being, sleep quality and satiety, and lower values indicate positive effects on the scales for hunger and for interest in food:

Mood: 0=poor, 15=excellent.
Vitality (feeling of energy): 0=poor, 15=excellent.
Well-being: 0=poor, 15=excellent.
Sleep quality: 0=poor, 15=excellent.
Hunger: 0=not at all hungry, 15=very hungry.
Satiety: 0=do not feel full quicker, 15=feel full very quickly.
Interest in food: 0=not at all interested, 15=very much interested in food.

The results were tabulated as means±standard deviations (n=6):

| Parameter | Day 0 | Day 1 | Day 7 | Day 14 |
|---|---|---|---|---|
| Mood | 5.2 ± 1.5 | 7.7 ± 1.3 | 9.5 ± 1.7 | 10.3 ± 2.2 |
| Vitality | 4.8 ± 2.7 | 7.2 ± 1.9 | 9.5 ± 1.9 | 9.8 ± 2.3 |
| Wellbeing | 5.6 ± 2.1 | 8.1 ± 1.0 | 10.2 ± 1.7 | 9.9 ± 2.3 |
| Sleep quality | 4.3 ± 2.6 | 6.9 ± 2.5 | 7.7 ± 2.7 | 9.1 ± 2.5 |
| Hunger | 7.7 ± 3.0 | 5.6 ± 2.6 | 3.5 ± 2.1 | 3.8 ± 2.6 |
| Satiety | 6.8 ± 2.7 | 8.5 ± 1.8 | 9.7 ± 2.8 | 11.1 ± 2.4 |
| Interest in food | 8.4 ± 3.4 | 6.0 ± 2.1 | 2.5 ± 1.1 | 3.0 ± 2.4 |

It was concluded that the treatment improved mood, vitality, well-being and sleep quality, at the same time reducing hunger sensations, increasing the onset of satiety when eating, and significantly reduced interest in food, the last interpreted as an effect on the reward response, namely that the treatment elicited a reward response which almost completely substituted for the reward response created by food.

1. A method of modulating the dopaminergic system of the central nervous system, the method comprising administering to a subject a dopamine precursor and/or a dopamine agonist in an amount effective to induce a reward response in the subject.

2. The method of claim 1, wherein said reward response simulates a desired state of being in the subject.

3. The method of claim 3, wherein said reward response simulates a feeling of having already eaten.

4. The method of claim 1, wherein both a dopamine precursor and a dopamine agonist are administered to the subject.

5. The method of claim 4, wherein said dopamine precursor is L-DOPA.

6. The method of claim 4, wherein the dopamine agonist is an aporphine alkaloid.

7. The method of claim 4, wherein said dopamine precursor is L-DOPA and said dopamine agonist is an aporphine alkaloid.

8. The method of claim 4, wherein said L-DOPA and said aporphine alkaloid are derived from plants.

9. The method of claim 8, wherein said L-DOPA is extracted from Mucuna pruriens.

10. The method of claim 8, wherein said aporphine alkaloid is extracted from Nelumbo nucifera.

11. The method of claim 3, wherein said administration is oral and occurs within 90 minutes before the subject eats a meal.

12. The method of claim 3, comprising administering L-tyrosine (USP), an extract from Mucuna pruriens, an extract from Nelumbo nucifera, an extract from Citrus aurantium and an extract from Griffonia simplicifolia.

13. The method of claim 3, comprising administering an extract Mucuna pruriens, an extract from Nelumbo nucifera, and an extract from Camellia sinensis.

14. A composition for reducing a subject's desire to eat comprising an effective amount of a dopamine precursor, a dopamine agonist and pharmaceutically acceptable excipients.

15. The composition of claim 14, comprising L-tyrosine (USP), an extract from Mucuna pruriens, an extract from Nelumbo nucifera, an extract from Citrus aurantium and an extract from Griffonia simplicifolia.

16. The composition of claim 15, wherein said composition comprises 150 mg of L-tyrosine (USP), 100 mg of extract from Mucuna pruriens, 100 mg of leaf extract from Nelumbo nucifera, and 40 mg of extract from Citrus aurantium, wherein 25% by weight of said Mucuma pruriens extract is L-DOPA, 8% by weight of said Nelumbo nucifera leaf extract is aporphine alkaloids, 30% by weight of said Citrus aurantium is one or more of an alkaloid selected from the group consisting of synephrine, octopamine, hordenine, tyramine and N-methyl-tyramine, and 25% by weight of said Griffonia simplicifolia extract is 5-hydroxytryptophan.

17. The composition of claim 16, which is in capsule form.

18. The composition of claim 14 comprising an extract from Mucuna pruriens, an extract from Camellia sinensis, and a leaf extract from Nelumbo nucifera.

19. The composition of claim 18, wherein said composition comprises 300 mg of Mucuna pruriens, 250 mg of Camellia sinensis and 200 mg of Nelumbo nucifera, wherein 25% by weight of said Mucuna pruriens is L-DOPA, 36% by weight of said extract from Camellia sinensis is caffeine and 45% by weight is catechols, and 8% by weight of said Nelumbo nucifera leaf extract is aporphine alkaloids.

20. The composition of claim 19, which is in the form of a tablet.
